(19) United States
(12) Patent Application Publication
(10) Pub. No.: US 2020/0046519 A1
(43) Pub. Date: Feb. 13, 2020

(54) PERSONALIZED PRECISION SURGICAL POSITION SYSTEMS FOR ACETABULAR IMPLANT PROCEDURES AND METHODS FOR USE

(71) Applicant: Advanced Ortho-Med Technology, Inc., Castro Valley, CA (US)

(72) Inventor: Sok Gek Lim, Castro Valley, CA (US)

(21) Appl. No.: 16/322,648

(22) PCT Filed: Aug. 11, 2017
(86) PCT No.: PCT/US2017/046627

§ 371 Date: Feb. 1, 2019

(51) Int. Cl. A61F 2/46; A61B 17/17; A61B 34/10

(52) U.S. Cl. CPC A61F 2/4609; A61B 17/1746; A61B 2034/105; A61F 2002/4687; A61B 2034/108; A61B 34/10

(57) ABSTRACT

A precision positioning system for acetabular implants is personalized to fit a particular patient using 3D medical imagery, attaches to the patient and uses predetermined positioning slots to give a surgeon a wide variety of options in choosing where and how to stabilize various surgical tools during a medical procedure.

### CROSS REFERENCE TO RELATED APPLICATIONS

This Application claims priority to U.S. Provisional Patent Application No. 62376046 filed. Aug. 17, 2016 in United State Patent and Trademark Office (USPTO).

### INTRODUCTION

The present teachings provide a method for the construction of personalized patient and instrument systems for the precision positioning of acetabular implants. Surgeons have previously relied solely on their own judgement and skill in placing the implants and existing surgical guide systems are disfavored by doctors as they require long setup times and are costly to buy and maintain.

Therefore, there is need for a new system of precision positioning surgical instruments during acetabular implant procedures that is personalized fitted to the patient and that is quick and easy to setup and adjust. The following presents an invention intended to satisfy these needs.

### BRIEF SUMMARY OF THE INVENTION

The present invention provides a personalized design and 3D printing surgical guide for positioning an acetabular implant. The present teachings provide a method for the construction of personalized device for the precision positioning of acetabular implants. The method includes devising a personalized pre-surgical plan from the patient's radiographic images, including, but not limited to X-rays, CT and MR images and choice of commercially available surgical instruments from a range of manufacturers for use in the surgery.

The process for the personalized pre-surgical plan comprises the following steps: a 3D modelling module that constructs a 3D model of the patient's acetabulum from the radiographic images; a quantification module for obtaining the required measurements from the 3D model including, the best fit acetabular cup size and pre-surgical plan for reaming and inserting the acetabular implant; a designing module of the personalized devices from the 3D model of the patient and specifications of the surgical instruments; and a manufacturing module to produce the personalized devices, including, but not limited to 3D printing technology of these personalized devices and sterilized for use during the surgery. The personalized device is patient-personalized, easy to use, independent of patient positioning on the surgical table and the surgical table positioning.

According to various embodiments, a surgical positioning system, comprises a patient-personalized engaging base constructed from a 3D model that is reconstructed from CT, MR or similar images and that is conformed to and engages part of the rim of the acetabulum of the patient's pelvis, and one or more positioning stabilizers; said stabilizers each consisting of a pillar and a clamp that attach to said personalized engaging base to engage surgical instruments and align them in the correct position and at the correct angle for surgery.

According to various embodiments, the patient personalized engaging base can possess a flat top surface possessing holes for the insertion of attachment devices and possessing positioning slots for the insertion of the positioning stabilizers.

According to various embodiments, the attachment devices can be bone nails.

According to various embodiments, the positioning slots are designed to allow for the positioning stabilizer to be inserted and removed without additional attachment mechanisms.

According to various embodiments, the positioning slots are positioned to locate the positioning stabilizer in a position determined by a surgeon where it does not interfere with or obstruct the use of a surgical instrument during an operation.

According to various embodiments, the pillar can be shaped such that it can inserted into the positioning slots of the personalized engaging base.

According to various embodiments, the invention can further comprise a means for controlling the depth of a surgical instruments used in conjunction with said surgical positioning system.

According to various embodiments, the means for controlling the depth of surgical instruments can be a visible indicator that is applied to the surgical instruments.

According to various embodiments, the means for controlling the depth of surgical instruments is a physical block attached to the surgical instruments.

According to various embodiments, a method for using the surgical positioning system comprises providing a surgical positioning system, securing its engaging surface to a surgical patient; attaching one or more stabilizers to the engaging surface and using the stabilizer to stabilize one or more surgical instruments.

### BRIEF DESCRIPTION OF FIGURES

The present teachings will become more fully understood from the detailed description and the accompanying drawings, wherein:

FIG. 1 is a perspective, exploded view of the personalized device and its relation to the acetabulum.

FIG. 2 is a side view of an exemplary personalized engaging base of a version of the present invention.

FIG. 3 is a top view of an exemplary personalized engaging base of a version of the present invention.

FIG. 4 is a perspective view from above of an exemplary personalized engaging base of a version of the present invention.

FIG. 5 is a perspective view from above of an exemplary positioning stabilizer of a version of the present invention.

FIG. 6 is a perspective view from above of an exemplary depth controller of a version of the present invention.

FIG. 7 is a perspective view from above of an exemplary depth controller, after having been applied to a surgical tool.

### DETAILED DESCRIPTION OF THE INVENTION

In the Summary of the Invention above and in the Detailed Description of the Invention, and in the claims below, and in the accompanying drawings, reference is made to particular features (including method steps) of the invention. IT is to be understood that the disclosure of the invention in this specification includes all possible combinations of such particular features. For example, where a particular feature is disclosed in the context of a particular aspect or embodiment of the inventio, or a particular claim, that feature can also be used, to the extent possible, in combination with and/or in the context of other particular aspects and embodiments of the invention, and in the invention generally.

The term “comprises” and its grammatical equivalents thereof are used herein to mean that other components, ingredients, steps, etc. are optionally present. For example, an article “comprising” (or “which comprises”) components A, B, and C can consist of (i.e. contain only) A, B, and C, or can contain not only components A, B, and C but also one or more other components.

When reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having “an upper limit or no upper limit, depending of the variable being defined). For example, “at least 1” means 1 or more than 1. The term “at most” followed by a number is used herein to denote the end of a range ending with that number (which may be a range having 1 or 0 as it's lower limit, or a range having no lower limit, or a range having no lower limit, depending on the variable being defined). For example, “at most 4” means 4 or less than 4, and “at most 40%” means /10?/k or less than 40%. When, in this specification, a range is given as “(a first number) to (a second number)” or “(a first number)−(a second number),” this means a range whose lower limit is the first number and whose upper limit is the second number. For example, 25 to 100 mm means a range whose lower limit is 25 mm, and whose upper limit is 100 mm.

### Definitions:

### “CT” IS COMPUTERIZED TOMOGRAPHY AND GRAMMATICAL EQUIVALENTS;

### “MW” IS MAGNETIC RESONANCE AND GRAMMATICAL EQUIVALENTS;

The following description is exemplary in nature and is not intended to limit the present teachings, applications and uses in any way.

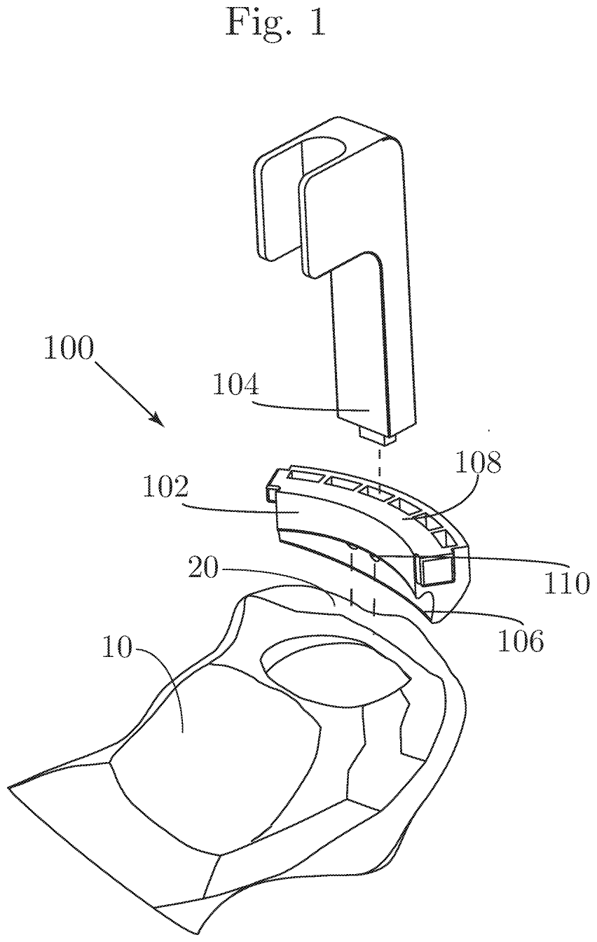

The teachings provide a method that includes devising a personalized pre-surgical plan to design and produce a personalized device for precision positioning of acetabular implants. FIG. 1 shows an exemplary removable personalized surgical positioning system 100 according to the present invention. It features two detachable components, the personalized engaging base 102 and the positioning stabilizer 104. The personalized device 100 is designed from the patient's radiographic images; choice of the surgical instruments to be used in the surgery, including, but not limited to reamers, impactors and acetabular cup insertion devices; and manufactured using 3D printing technology or any other suitable manufacturing process.

The process for the pre-surgical plan comprises the following steps: a 3D modelling module that constructs a 3D model of the patient's acetabulum from the radiographic images, including, but not limited to X-rays, CT and MR images; a quantification module for obtaining the required measurements from the 3D model including, but not limited to, the diameter of the acetabulum and the centroid of the acetabulum, and the anteversion and abduction angles, in order to find the best fit acetabular cup size and precision positioning for reaming and inserting the acetabular implant; a designing module of the personalized devices from the 3D model of the patient and specifications of the surgical instruments, the design of the personalized devices is based on quantitative measurements such as the anteversion and abduction angles, and the dimensions of the chosen surgical instruments to ensure that the surgery conforms to the pre-surgical plan, suitable and related acetabular landmarks are also used for the design of the personalized device; and a manufacturing module to produce the personalized devices, including, but not limited to 3D printing technology of these personalized devices and sterilized for use during the surgery. The personalized device is patient-personalized, easy to use, independent of patient positioning on the surgical table and the surgical table positioning.

FIG. 1 is an exemplary diagram showing how the surgical positioning system 100 can be attached to the acetabulum 10 of a patient. The 3D model of the patient's acetabulum 10 is reconstructed from the patient's radiographic images which are used in the development of a pre-surgical plan. The personalized engaging base 102 features a lower engaging surface 106 that is shaped and closely fit to the acetabulum 10 so that it conforms to the acetabulum rim 20. The personalized engaging base 102 can be designed to any shape that suits the surgeon's requirements including, but not limited to, the C-shape in FIG. 1. The shape and length of the engaging base 102 must be suitably designed so that it does not obstruct the operation in any way during the surgery.

The upper surface 108 of the personalized engaging base 102 is a flat surface and is designed to be perpendicular to the direction determined by, and calculated from, the anteversion and abduction angles for reaming the acetabulum and inserting the acetabular cup in the correct position. The anteversion and abduction angles can be specified by the surgeons or calculated from the 3D model to find the best position.

The personalized engaging base 102 is temporarily attached to the bone surrounding the acetabulum 20 using the holes 110 for the insertion of bone nails or other attachment devices.

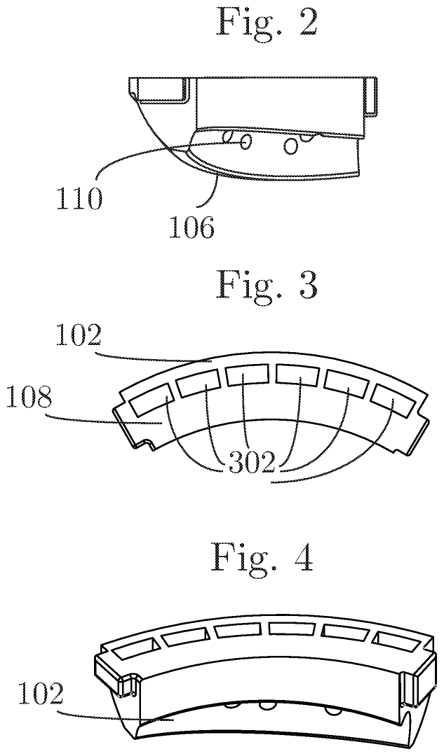

FIG. 3 is a view of the upper surface 108 of the personalized engaging base 102, showing the positioning slots 302 wherein the positioning stabilizer 104 is inserted. The positioning slots 302 provide flexibility and feasibility of the use of the personalized device 100 where the surgeon can place the positioning stabilizer 104 at a position that does not interfere with or obstruct the use of the surgical instrument during the operation.

The positioning stabilizer 104 in FIG. 5 consists of a pillar 502, which slots into the personalized engaging base and a clamp 504 that engages a surgical instrument. The positioning stabilizer 104 is designed to position the surgical instruments at the correct alignment with respect to the anteversion and abduction angles and the clamp 504 which attach and stabilize the surgical implant instruments including, but not limited to, reamers, impactors and acetabular cup insertion tools.

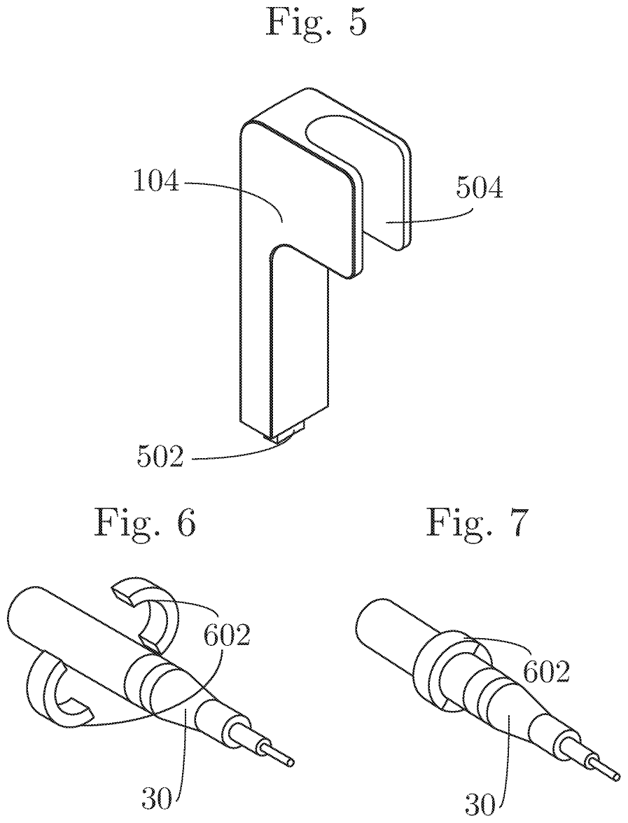

As shown in FIG. 6, the system also features a depth controller 602 to prevent over/under reaming of the acetabulum 20. The depth controller 602 can be a ring structure that attaches to a surgical instrument 30 by physically blocking the surgical tool from moving through the positioning stabilizer 104 completely. It can also be a visual indicator applied to the surgical tool, giving the user a visual mark for when to stop lowering the surgical instrument 30.

The clamp 504 can be designed to fit and stabilize from a plurality of commercially available reamers with different cross-sectional shapes and sizes.

The clamp 504 can be designed to fit and stabilize from a plurality of commercially available impactors and acetabular cup inserter with different cross-sectional shapes and sizes.

The surgical instruments to be used during the surgery are chosen by the surgeon during the development of the pre-surgical plan. The surgeon may choose to employ instruments from a plurality of manufacturers with different cross-sectional shapes and sizes. Depending on the choice of instruments, it may be necessary to design more than one clamp 504 to fit the dimensions of the various instruments.

The personalized engaging base 102 and the positioning stabilizer(s) 104 are designed using CAD software and realized using 3D printing machinery or other suitable manufacturing technologies. After realization, they are sterilized prior to use in surgery.

During the surgery, the personalized engaging base 102 is attached to the bone of the acetabulum 20 using bone nails inserted through the holes 110. The positioning stabilizer 104 is inserted in the slots 302 in the upper surface 108 of the personalized engaging base 102. Surgical implant tools including, but not limited to, reamers, impactors and acetabular cup insertion tools are aligned by the positioning stabilizer 104 while they are used to ream the acetabulum 20 and insert the acetabular cup. If a plurality of positioning stabilizers 104 is required during the surgery, each one is inserted as required and removed after it is used to position one or more surgical instruments. A plurality of stabilizers can also be used at the same time.

The parts of the system used during surgery can be made of sterile materials, suitable for use in a surgical environment such as, but not limited to, plastic or surgical stainless steel.

Although the present invention has been described in considerable detail with reference to certain preferred versions thereof, other versions are possible. Therefore, the spirit and scope of the appended claims should not be limited to the description of the preferred versions contained herein.

I claim:

1. A personalized surgical positioning system, comprising:
a personalized patient engaging base constructed from a 3D model that is reconstructed from CT, MR or similar images and that is conformed to and engages part of the rim of the acetabulum of a specific pelvis; and one or more positioning stabilizers; said stabilizers each consisting of a pillar and a clamp that attach to said personalized engaging base to engage surgical instruments and align them in the correct position and at the correct angle for surgery.

2. The personalized surgical positioning system of claim 1, wherein, said personalized patient engaging base possesses a flat top surface possessing holes for the insertion of attachment devices and possessing positioning slots for the insertion of said positioning stabilizers.

3. The personalized surgical positioning system of claim 2, wherein said attachment devices are bone nails.

4. The personalized surgical positioning system of claim 2, wherein said positioning slots are designed to allow for said positioning stabilizer to be inserted and removed without any additional attachment mechanisms.

5. The personalized surgical positioning system of claim 2, wherein said positioning slots are positioned to locate said positioning stabilizer in a position determined by a surgeon where it does not interfere with or obstruct the use of a surgical instrument during an operation.

6. The personalized surgical positioning system of claim 2, wherein said pillar can be inserted into said positioning slots of said personalized patient engaging base.

7. The personalized surgical positioning system of claim 2, further comprising a means for controlling the depth of a surgical instruments used in conjunction with said surgical positioning system.

8. The personalized surgical positioning system of claim 7, wherein said means for controlling the depth of surgical instruments is a visible indicator that is applied to said surgical instruments.

9. The surgical positioning system of claim 7, wherein said means for controlling the depth of surgical instruments is a physical block attached to said surgical instruments.

10. A method for using the device of claim 1 comprising the following steps:
a. providing one or more surgical positioning system of claim 1;
b. securing said personalized engaging surface to a surgical patient;
c. attaching one or more stabilizers to said personalized engaging surface;
d. use said stabilizers to stabilize one or more surgical instruments including, but not limited to, reamers, impactors and acetabular cup implant tools.
